(19) United States
(12) Patent Application Publication
(10) Pub. No.: US 2007/0011773 A1
(43) Pub. Date: Jan. 11, 2007

(54) Method of resistance management for transgenic crops

(75) Inventors: Martin Stephen Clough, Bracknell (GB); Alan Roy McCaffery, Bracknell (GB)

Correspondence Address: DANA REWOLDT;GARST SEED COMPANY, 2369 330TH STREET, PO BOX 500, SLATER, IA, 50244, US

(73) Assignee: Syngenta Limited

(21) Appl. No.: 10/551,894

(22) PCT Filed: Mar. 3, 2004
(86) PCT No.: PCT/GB04/00901

§ 371 Date: Aug. 11, 2006

(30) Foreign Application Priority Data
Apr. 4, 2003 (GB) 0307871.4
Aug. 18, 2003 (GB) 0319372.9

(51) Int. Cl. A01H 1/00; A01H 3/00

(52) U.S. Cl. 800/279; 47/58.1 R

(57) ABSTRACT

The present invention provides a method for reducing the incidence of resistance of pests to pesticidal plants. In particular, there is provided a locus at which plant pests feed comprising at least two regions, characterised in that: a) a first region comprises plants which produce at least a first pesticidal toxin; and b) a second region comprises plants which produce at least a second pesticidal toxin; wherein a pest which can develop resistance to the first toxin does not develop resistance to the second toxin, and the first region comprises plants which produce the first toxin but not the second toxin when the plants of the second region produce the second toxin but not the first toxin. In one aspect of the invention, the plant pests are insects. The invention also provides a method for controlling insects.

The present invention relates to a method for preventing or reducing the incidence of pest resistance to pesticidal plants. In particular, it relates to preventing resistance of insects to transgenic insecticidal plants such as cotton from being spread through an insect population.

Millions of hectares of crops worldwide are damaged each year as a result of attack by insect pests. Controlling insect pests is a serious problem for farmers who look to minimise such crop damage and resulting yield losses. This is especially true in cotton, a crop of great commercial importance. Thousands of hectares of cotton crops are damaged by a wide range of insect pests each year.

Entomologists calculate that crop damage caused by insects has doubled in the last 50 years, related to intensified farming efforts to feed a growing world population. The agrochemical industry has tried to control this problem with chemical solutions and insecticide spraying has become a commonplace method adopted to minimise the damage to crops. Today there are over 200 different active ingredients, in some 40,000 commercial chemical products, all targeted at reducing insect damage.

During one growing season, many insecticide sprays may be applied to a single crop. This intensive use of chemical insecticides imposes a high selection pressure and without careful management can lead to the rapid build-up of resistance. Examples of insect resistance to pesticides have been documented worldwide. It is estimated that more than 500 arthropod pests worldwide have developed resistance to chemicals. In China, cotton yields fell by one-third between 1991 and 1993, largely due to the cotton bollworm (Helicoverpa armigera) which developed resistance to all the chemicals which were used for its control.

Insects have an exceptional ability to adapt to their environment. They have many mechanisms which allow the rapid build-up of resistance in an insect population, such as short life cycles, a high reproductive rate and the ability to travel long distances. Natural selection allows insects with resistance genes to survive, and the resistance trait is passed on to their offspring. Resistant insects continue to multiply as susceptible insects are eliminated by the pesticide, until eventually, insects that have a resistance gene(s) are predominant in the population and the pesticide is no longer effective. The speed with which resistance develops depends on many factors such as the rate of insect reproduction, the migration and host range of the insect, the persistence of the pesticide, the fitness costs of resistance and how often the pesticide is applied.

There are a number of known mechanisms of resistance in insects. For example, resistant insects may detoxify the toxin or remove it from their bodies faster than susceptible insects (metabolic resistance); the site where the toxin usually binds in the insect maybe modified to reduce this interaction (altered target-site resistance); resistant insects may absorb the toxin slower than susceptible insects (penetration resistance); or resistant insects may detect and avoid the toxin (behavioural resistance). Pests often use more than one of these mechanisms at the same time.

One solution aimed at reducing the number of insecticide sprays and managing insect resistance is to engineer crop plants to synthesise their own insecticide. Plants may be engineered to contain, for example, insecticidal genes from other organisms. Currently the most economically significant insecticidal transgenic plants are those which contain genes from the bacterium Bacillus thuringiensis that produce proteins that control Lepidopteran or Coleopteran pests. Not only can the development of transgenic crops reduce the use of broad-spectrum insecticides, but they also are more target-pest specific. This means that populations of beneficial insects may not be affected. Pest control is easier with transgenic crops because, using tissue specific promoters, the insecticidal toxin can be targeted to different parts of the plant, such as the roots, which are hard to spray with conventional pesticides.

However as transgenic plants may provide extensive and continuous selection pressure on pest populations, there can be a greater potential for resistance development than with conventional insecticides. Also, unlike chemical insecticides, there are still very few genes and proteins that are known to be effective for the protection of transgenic crops against insects. In fact, only a few transgenic crops have been commercialised to date, between them comprising only a handful of insecticidal genes. Therefore prevention and management of resistance build-up in populations of target insects is vitally important.

Insect Resistance Management (IRM) programs are designed to control such a build-up of resistance, and include the use of synthetic insecticides, biological insecticides, transgenic plants, beneficial insects, cultural practices, crop rotation, pest-resistant crop varieties and chemical attractants or deterrents.

One strategy employed in IRM programs is the use of refuges (refugia). In practice, refugia are often used in combination with a high dose of insecticidal agent, as currently mandated by some regulatory authorities. For transgenic crops, a refuge is the designation of a percentage of the cropped area as non-transgenic, with or without selected control treatments. Refuge areas may be within fields of transgenic or treated crops, around the border of such fields or even in adjacent fields depending on the biology of the target pests. Refugia assume and work best in situations where resistance is a recessive trait. Refugia serve to maintain a population of susceptible pests. When members of this susceptible population mate with any resistant insects that emerge from protected fields, their susceptible genes dilute any resistant genes in the overall population.

In essence, the use of refugia serves as a mechanism for producing a population of insect pests, pests which the growers are trying to control in their field. Resistant insects which survive the insecticidal effects of feeding on the transgenic crop are not killed.

Prior art in the field of insect resistance management for transgenic crops relates mainly to the use of a non-transgenic refuge surrounding a transgenic crop for reducing the incidence of resistance. For example, Tang et al. (2001) studied the resistance of Plutella xylostella when feeding on transgenic broccoli expressing Cry1Ac, and demonstrated that resistance to Cry1Ac could be delayed by increasing the proportion of non-transgenic refuge plants.

Roush (1997) describes four possible strategies for managing resistance to Bt-transgenic crops: (1) refuges of non-transgenic host plants preferably combined with high toxin expression in the crop plants, (2) moderate expression of toxin in crop plants to allow susceptible insects to survive, (3) use of different toxins deployed in different varieties in a mosaic in the same area, and (4) use of varieties where each plant has a mixture of toxins. Roush describes that mosaics are the worst way to deploy two toxicants, and that the most promising long term solution to resistance management is using ‘pyramided’ varieties containing two or more toxins. This differs from the present invention which does not include the use of mosaics. Further, although the present invention does not exclude the use of plants producing more than one insecticidal toxin, the invention per se does not relate to the use of pyramided varieties. In contrast to Roush, the present invention teaches that the use of two regions, each comprising plants producing different insecticidal toxins, is an effective tool in insect resistance management.

Driver et al. (U.S. Pat. No. 5,640,804) relates to the use of transgenic pest trap plants adjacent to agronomically important non-transgenic crop plants, such as walnut. The pest trap plants are a preferred host for the pests, and the underlying concept is to protect crop plants by attracting pest insects away from the crop plants, and further to provide an insecticidal toxin to kill the pest insects. This is different to the present invention wherein both plant types are transgenic, neither intended as a preferred pest host, with the aim of reducing the incidence of resistance.

Romano (U.S. Pat. No. 6,501,009) relates to the use of Cry3B and variants of Cry3B for controlling coleopteran pests. One aspect of this publication relates to an improved method of delaying the onset of resistance to Cry3B. This is achieved by using an optimised DNA cassette to achieve higher levels of expression of Cry3B and/or by simultaneously exposing target insects to both Cry3 toxins and other insecticidal proteins. Further, ‘simultaneous exposure’ refers to the expression of Cry3B and another insecticidal protein in the same plant using a gene stacking type approach. This is different to the present invention, where different insecticidal toxins are produced in different plants.

At its best, the current refuge strategy works well, but this may not be the case in all circumstances. For example, a refuge strategy works well when the resistance trait to be controlled is a recessive trait. However, if the resistance trait is dominant, the refuge will be much less effective at controlling resistance, and at best will only slowly dilute out the resistance trait. Also, refugia work best for IRM when combined with a crop expressing a high dose of insecticidal toxin so that all insects which are heterozygous for a resistance allele are killed. This is to ensure that the resistance trait is recessive in nature. However, it is often difficult to ensure expression of a sufficiently high enough dose of toxin to kill insects which are heterozygous for the resistance allele, and often difficult to ensure such high doses are expressed all season long.

The present patent application describes an improved method for IRM, which works to control recessive or dominant resistance traits better than using a refuge. This improved method may also be used in conjunction with a refuge. The invention uses a region comprising plants which produce at least one insecticidal toxin (principal crop plants), said toxin being different to the toxin produced by the plants in a second region. The effect of the different insecticidal toxins is to kill insects which are resistant to the toxin of the principal crop plants rather than allowing them to breed and thus spread the resistance trait. In this way insects are unlikely to survive as this would require resistance to at least two different insecticidal toxins preferably having different modes of action. Therefore the incidence of resistance is reduced. This invention may be used in conjunction with other IRM techniques.

According to the present invention there is provided a locus at which plant pests feed comprising at least two regions, characterised in that: a) a first region comprises plants which produce at least a first pesticidal toxin; and b) a second region comprises plants which produce at least a second pesticidal toxin; wherein a pest which can develop resistance to the first toxin does not develop resistance to the second toxin, and the first region comprises plants which produce the first toxin but not the second toxin when the plants of the second region produce the second toxin but not the first toxin. In one embodiment of the invention, the plant pests are selected from the group consisting of insects, mites and nematodes. In further embodiment of the invention, the plant pests are insects. In this aspect of the invention, the plants produce insecticidal toxins.

The word insecticidal as used herein describes the effect of a toxin on insects. It is not limited to death of the insect, but also includes any effect which is detrimental to the insect, for example sickness, anti-feedant activity, growth retardation, reduced reproductive ability and reduced fecundity.

Resistant insects are those that do not suffer any substantial or appreciable detrimental effects as a result of exposure to or ingestion of a suitable dose of insecticidal toxin. A suitable dose of insecticidal toxin may be measured by exposure to or ingestion of the toxin by a susceptible insect and identification of the dose at which detrimental effect(s) are observed. Detrimental effects to the insect are described above in the definition of the word insecticidal. These detrimental effects will reduce the incidence of transfer of a resistance trait from a resistant insect to future generations of insects. In one aspect of the invention, the resistance trait is a dominant trait.

The word plants as used herein refers to plants and plant parts and includes seeds.

The invention includes plants which produce more than one toxin, for example via gene stacking. The plants of either the first and/or second region may even produce the same toxins, with the proviso that the first region comprises plants which produce the first toxin but not the second toxin When the plants of the second region produce the second toxin but not the first toxin. The invention is not limited to loci which comprise first and second regions that only comprise plants which produce insecticidal toxin(s), but may also contain other plants in addition. The other plants may be non-transgenic. In one aspect of the invention, the plants of either region may also produce toxins to make them resistant to non-insect pests such as viruses, fungi or nematodes. In another aspect of the invention, the plants of either region may be tolerant to chemical herbicides. In a further aspect of the invention, the locus may comprise more than two regions, wherein said additional regions may comprise plants which produce insecticidal toxins. In one embodiment of the invention, the locus may comprise a third region, which region comprises non-insecticidal plants.

The skilled man will be familiar with insects which feed at the locus. Preferably, insects which feed at the locus include pest insects which cause damage to plants. More preferably, this includes insects which are, or can develop to be, resistant to an insecticidal toxin. More preferably still, it includes insects selected from the group comprising: Acanthoscelides obtectus, Bruchus spp., Callosobruchus sps. (bruchid beetles), Agriotes spp. (wireworms), Amphimallon spp. (chafer beetles), Anthonomus grandis (cotton boll weevil), Ceutorhynchus assimilis (cabbage seed weevil), Cylas spp. (sweet potato weevils), Diabrotica spp. (corn root worms), Epicauta spp. (black blister beetles), Epilachna spp. (melon beetles etc.), Leptinotarsa decemlineata (Colorado potato beetle) Meligethes spp. (blossom beetles), Melolontha spp. (cockchafers), Phyllotreta spp., Psylliodes spp. (flea beetles), Popillia japonica (Japanese beetle), Scolytus spp. (bark beetles), Acleris spp. (fruit tree tortrix), Acraea acerata (sweet potato butterfly), Agrotis spp. (cutworms), Autographa gamma (silver-Y moth), Chilo spp. (stalk borers), Cydia pomonella (codling moth), Diparopsis spp. (red bollworms), Ephestia spp. (warehouse moths), Heliothis spp., Helicoverpa spp. (bollworms, bollworms), Mamestra brassicae (cabbage moth), Manduca spp. (hornworms), Maruca testulalis (mung moth), Mythimna spp. (cereal armyworms), Ostrinia nubilalis (European corn borer), Pectinophora gossypiella (pink bollworm), Phthorimaea operculella (potato tuber moth), Pieris brassicae (large white butterfly), Pieris rapae (small white butterfly), Plodia interpunctella (Indian grain moth), Plutella xylostella (diamond-back moth), Pseudoplusia includens (soybean looper), Sitatroga cerealella (Angoumois grain moth), Spodoptera spp. (armyworms), Trichoplusia ni (cabbage semilooper), Acheta spp. (field crickets), Gryllotalpa spp. (mole crickets), Locusta migratoria (migratory locust), Schistocerca gregaria (desert locust), Acrythosiphon pisum, Drosophila spp., Acrosternum hilare (green stink bug), Aphis gossypii (cotton aphid), Campylomma liebnechti (apple dimpling bug), Creontiades dilutus (green mind), Crocidosema plebejana (cotton tipwormn), Earias huegelli (rough bollworm), Euschistus servus (brown stink bug), Frankliniella spp. (thrips), Lygus lineolaris (tarnished plant bug), Tetranychus urticae (spider mite) and Thrips tabaci (onion thrips).

Most preferably, it includes insects selected from the group comprising: Anthonomus grandis (cotton boll weevil), Pectinophora gossypiella (ink bollworm), Heliothis virescens (tobacco budworm), Helicoverpa zea (cotton bollworm), Helicoverpa armigera (American bollworm), Helicoverpa punctigera (native bollworm), Spodoptera exigua (beet armyworm) and Spodoptera frugiperda (fall armyworm).

With the benefit of the present disclosure, the skilled man will be familiar with insecticidal toxins that can be expressed in plants which may be suitable for use in this invention. Suitable toxins may even be those known in the prior art. For example, they include crystal proteins from Bacillus thuringiensis, many of which are have been extensively studied and are well known in the prior art such as Cry1Ac, Cry2Ab and Cry1F. A non-limiting list of protein toxins from Bacillus thuringiensis which may be used in the present invention is available on the internet at http://www.biols.susx.ac.uk/home/Neil_Crickmore/Bt/index.html. Further non-limiting examples of insecticidal toxins are vegetative insecticidal proteins VIP3A and VIP3B from Bacillus thuringiensis, 445 from Paecilomyces farinosus (see International Patent Application publication number WO01/00841) and GGK (see International Patent Application publication number WO02/098911). Alternative suitable insecticidal toxins may, for example, be isolated from bacteria, fungi, plants or other sources. The genes encoding these toxins can be cloned and transformed into suitable plants under the control of a plant-operable gene cassette, using standard molecular and cell biology techniques. The toxins may be targeted to particular parts of the plant such as the roots, leaves or seeds by cloning the genes encoding the toxins to be under the control of tissue-specific promoters. Alternatively, the toxins may only be produced at a certain growth stage of the plant through use of inducible or temporal promoters. The first and second insecticidal toxins may be insecticidal towards different spectra of insect species. Preferably the first and second toxins are insecticidal towards the same or similar insect species, or overlapping spectra of insect species. Preferably the first and second toxins act at different binding sites to one another. More preferably, the first and second toxins have different modes of action to one another.

In one aspect of the invention, the plants of the first and second regions may optionally exhibit other beneficial traits, which also may have been introduced via gene cloning and plant transformation. Any number of these traits may be stacked with the insecticidal toxin in the plants. For example, the plants of either region may exhibit resistance to a particular herbicide, fungal disease, viral infection or nematode infestation. An example of resistance to a herbicide is described in International Patent Application publication number WO 00/66747 wherein a mutant form of the enzyme EPSP synthase is expressed in a plant so that the plant is tolerant to the herbicide glyphosate.

In a particular embodiment of the present invention, the second region is within a mile from the first region. In another embodiment of the present invention, the second region is within a quarter of a mile from the first region. In a further embodiment the second region is adjacent to the first region. In a still further embodiment the second region is a border around the perimeter of the first region. In a further embodiment the second region comprises one or more strips within the first region. In a further embodiment the locus comprises a random distribution of first and second regions within the locus. The schematic diagrams provided in FIGS. 1 to 10 represent non-limiting examples of the possible arrangement of first and second regions within the locus of the present invention. In one aspect of the invention, a mosaic pattern of first and second regions may be specifically excluded. In an embodiment where the locus comprises a third region, which region comprises non-insecticidal plants, said third region may also be arranged with respect to the first and second regions according to the arrangements described above.

A locus according to the present invention may be comprised by a farm, wherein the at least two regions are fields. The following non-limiting examples describe possible arrangements of the fields. For example, the fields may be adjacent to one another (see FIGS. 5 to 7). Alternatively, the locus of the present invention may be a field, wherein the at least two regions are areas of the field comprising different plants. The second region may be arranged, for example, as a border around the perimeter of the first region (see FIG. 1), as a series of horizontal or vertical strips amongst the first regions (see FIGS. 2 and 3) or as a block within the first region (see FIG. 4). The deployment of the regions within the locus is dependent on the biology or mobility of the target pest. In one aspect of the invention, there may be a border around the locus comprising or consisting of non-insecticidal plants, non-transgenic plants, non-host plants or being uncultivated. In another aspect of the invention, the locus may comprise a third region, said region being, for example, a border around the first and/or second regions, and comprising non-insecticidal plants.

Alternatively, a locus according to the present invention may be comprised by, for example, a garden, forest, glasshouse or seed store. The locus may even be comprised by a lake such that the invention is used-to control aquatic insects. However, the scope of this invention is clearly restricted to loci wherein the invention would be functional. The skilled man would understand that the present invention excludes the possibility of the locus being the world, wherein the first region is America and the second region is Europe, for example.

In a particular embodiment of the present invention the locus comprises at least two regions wherein the first region comprises plants which produce at least a first insecticidal toxin, and the second region comprises plants which produce at least a second insecticidal toxin, wherein the first insecticidal toxin has a different mode of action to the second insecticidal toxin. Examples of known modes of action of insecticidal chemicals and toxins include, but are not limited to acetyl choline esterase inhibitors, GABA-gated chloride channel antagonists, sodium channel modulators, acetyl choline receptor modulators, chloride channel activators, juvenile hormone mimics, fumigants, selective feeding blockers, growth inhibitors, disrupters of insect midgut membranes, inhibitors of oxidative phosphorylation, disrupters of ATP formation, oxidative phosphorylation uncouplers, inhibitors of magnesium stimulated ATPase, inhibitors of chitin biosynthesis, ecdysone agonist or disrupters, electron transport inhibitors and voltage dependant sodium channel blockers. For example, the first toxin may be a disrupter of the insect midgut membrane, such as a crystal toxin from Bacillus thuringiensis, and the second toxin may be a growth inhibitor.

In a further embodiment of the invention, the first insecticidal toxin is a crystal protein from Bacillus thuringiensis and the second insecticidal toxin is a vegetative insecticidal protein (VIP) from Bacillus thuringiensis, or vice versa Many crystal proteins from Bacillus thuringiensis have been isolated and are known to have an insecticidal effect. Preferably, the crystal protein of the present invention is Cry1Ac. Preferably the VIP protein is VIP3A. In one aspect of the invention, the plants which produce either the first or second insecticidal toxin may comprise both Cry1Ac and Cry2Ab.

In a further embodiment of the present invention, the plants which comprise the first toxin and the plants which comprise the second toxin are from different genera. For example, the plants which comprise the first toxin may be cotton plants from the genus Gossypium L., and the plants which comprise the second toxin may be corn plants from the genus Zea L. In further non-limiting example, the plants which comprise the first toxin may be wheat plants from the genus Triticum L. and the plants which comprise the second toxin may be barley plants from the genus Hordeum L.

In a further embodiment of the present invention, the plants which comprise the first toxin and the plants which comprise the second toxin are from the same genus. In a farther embodiment of the present invention, the plants which comprise the first toxin and the plants which comprise the second toxin are cotton plants. Preferably the plants are of the same species. More preferably the plants are the Upland Cotton species, Gossypium hirsutum. The cotton plants of the first and second regions may be the same or different varieties.

In a further embodiment of the invention, at least 5% of the locus comprises the first region and at least 5% of the locus comprises the second region. In one alternative, at least 20% of the locus comprises the first region and at least 20% of the locus comprises the second region. In another alternative, 50% of the locus comprises the first region and. 50% of the locus comprises the second region.

Further, the invention provides the option of applying a chemical spray to some or all of the regions or parts of said regions within the locus. The chemical may, for example, be an insecticide, fungicide or herbicide. Preferably such an insecticidal chemical acts at a different binding site to the insecticidal toxins produced by the plants of the first and/or second regions. Preferably such an insecticidal chemical has a different mode of action to the insecticidal toxins produced by the plants of the first and/or second regions. More preferably, the chemical is not a Bacillus thuringiensis insecticide.

According to the present invention there is provided a method of controlling insects comprising providing a locus at which insects feed comprising at least two regions, characterised in that: a) a first region comprises plants which produce at least a first insecticidal toxin; and b) a second region comprises plants which produce at least a second insecticidal toxin; wherein an insect which can develop resistance to the first toxin does not develop resistance to the second toxin, and the first region comprises plants which produce the first toxin but not the second toxin when the plants of the second region produce the second toxin but not the first toxin.

The words ‘controlling’ or ‘control’ as used herein refer not just to death of insects, but also include other detrimental effects on insects such as sickness, anti-feedant activity, growth retardation, reduced reproductive ability and reduced fecundity.

In an embodiment of the invention, a locus as described above is used in a method of controlling insects.

According to the present invention there is provided a method of reducing the incidence of resistance to a first insecticidal toxin comprising the steps of providing a locus at which insects feed comprising at least two regions, characterised in that: a) a first region comprises plants which produce at least a first insecticidal toxin; and b) a second region comprises plants which produce at least a second insecticidal toxin; wherein an insect which can develop resistance to the first toxin does not develop resistance to the second toxin, and the first region comprises plants which produce the first toxin but not the second when the plants of the second region produce the second toxin but not the first, so that insects which have developed or are developing resistance to the first insecticidal toxin are controlled by the second toxin.

The terms ‘developed resistance’ and ‘developing resistance’ refer to resistance within a population of insects rather than individual insects. While it may be possible for an individual insect to apparently become resistant to an insecticidal toxin, for example by the overproduction of a detoxification enzyme in response to ingestion of the toxin, this ability to induce enzyme production is likely pre-determined as a result of a mutation in the insect genome. Therefore, insects hatch as either resistant or susceptible to a particular toxin. The terms ‘developed resistance’ or ‘developing resistance’ encompass the development of resistance via evolution through generations of breeding.

In an embodiment of the invention, a locus as described above is used in a method of reducing the incidence of resistance of insects to a first insecticidal toxin.

In a further embodiment of the present invention, there is a method as described above or a locus as described above wherein either the first or second region comprises Bollgard® cotton plants. Preferably either the first or second region comprises Bollgard I® cotton plants which produce the insecticidal toxin Cry1Ac. More preferably either the first or second region comprises Bollgard II® cotton plants which produce the insecticidal toxins Cry1Ac and Cry2Ab in the same plant.

In a further embodiment of the present invention, there is a method as described above or a locus as described above wherein the first region comprises Bollgard® cotton plants and the second region comprises VIP cotton plants. In a further embodiment still, there is a method as described above or a locus as described above wherein the first region comprises VIP cotton plants and the second region comprises Bollgard® cotton plants. The Bollgard® cotton plants may be Bollgard I® or Bollgard II®. The VIP cotton plants produce the insecticidal toxin VIP3A (see, for example, International Patent Application Number PCT/EP03/11725). In a further aspect of the invention, the first region comprises VIP cotton plants and the second region comprises Bollgard® cotton plants.

In a further embodiment of the present invention, there is provided a method or a locus as described above, wherein the first or second region comprises plants expressing the insecticidal toxin Cry3A or a modified version thereof. In a further embodiment of the present invention, there is provided a method or a locus as described above, wherein the first region comprises plants which comprise Cry3A toxin from Bacillus thuringiensis or a modified version thereof, and the second region comprises plants which comprise Cry3B toxin from Bacillus thuringiensis. Modified versions of Cry3A include, for example, Cry3A toxins comprising a cathepsin G protease recognition site in domain 1, such as those described in International Patent Application publication number WO03/18810.

In addition to loci at which insects feed comprising plants expressing insecticidal toxins for reducing the incidence of resistance in insects, the present invention further includes loci at which nematodes or mites feed, comprising plants expressing nematicidal or miticidal proteins for reducing the incidence of resistance in nematodes or mites respectively, and methods and uses thereof.

In one aspect of the present invention neither the plants of the first region, nor the plants of the second region produce a Cry3 toxin. In a further aspect of the present invention neither the plants of the first region, nor the plants of the second region produce a Cry3B toxin. For example, the present invention may specifically exclude a locus wherein one of the regions of said locus comprises plants which produce a Cry3B toxin or variants thereof

### DESCRIPTION OF THE FIGURES

The accompanying figures illustrate non-limiting examples of the arrangement of the first and second regions within the locus.

FIG. 1—Second region forms a border around the perimeter of the first region

FIG. 2—Second region forms brackets either side of the first region

FIG. 3—Second region forms a series of horizontal or vertical strips amongst the first regions

FIG. 4—Second region forms a block within the first region

FIGS. 5-7—First and second regions are adjacent

FIG. 8-10—A plurality of first and second regions

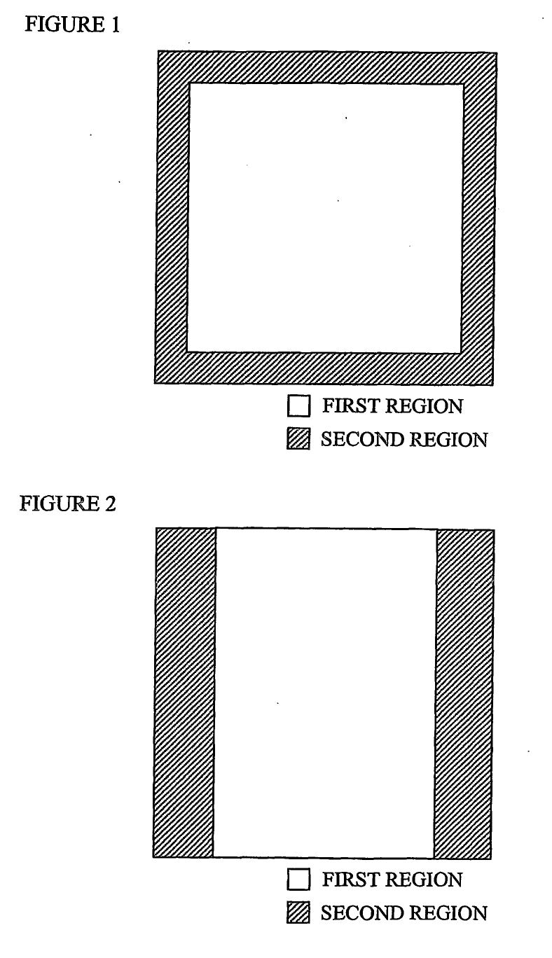

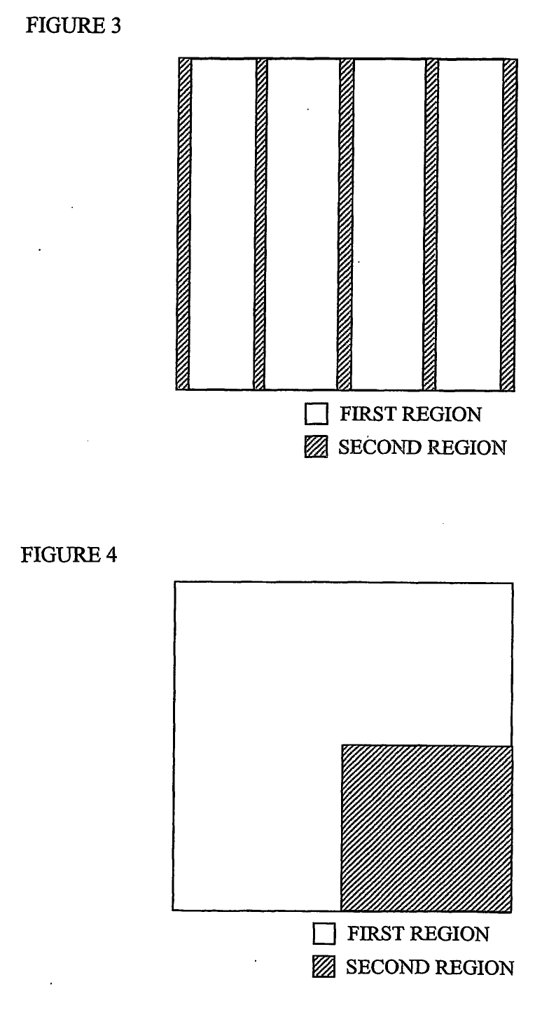

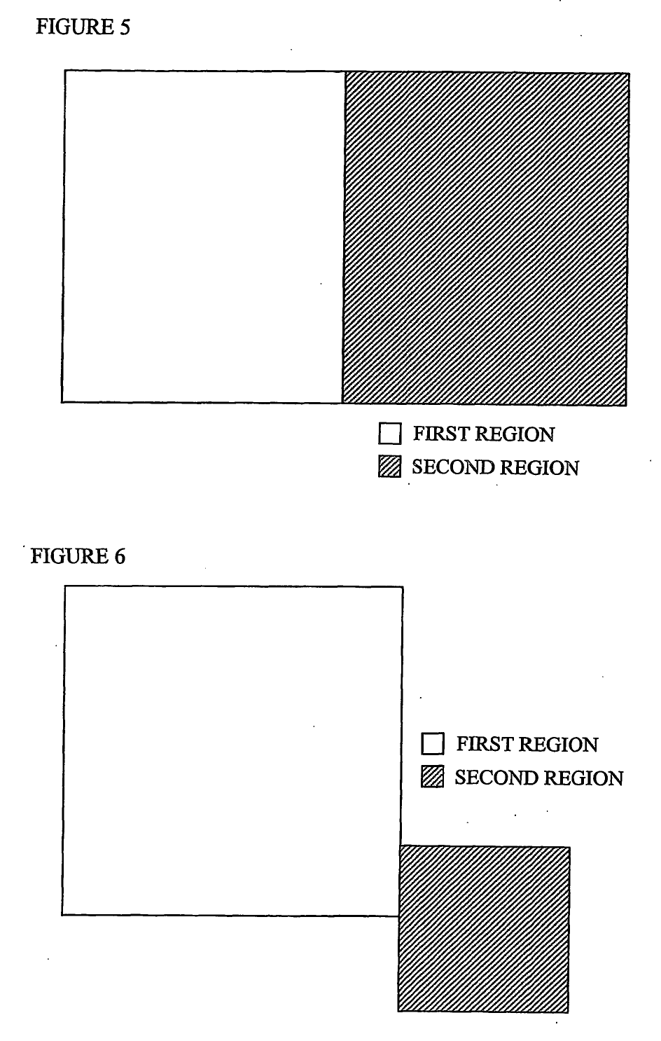

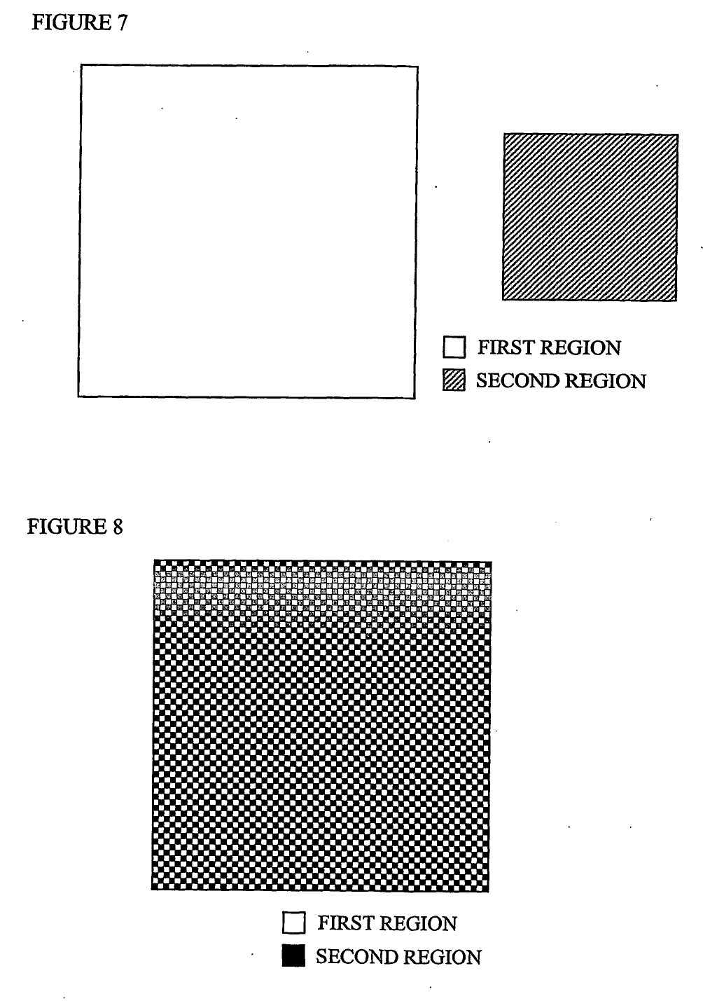

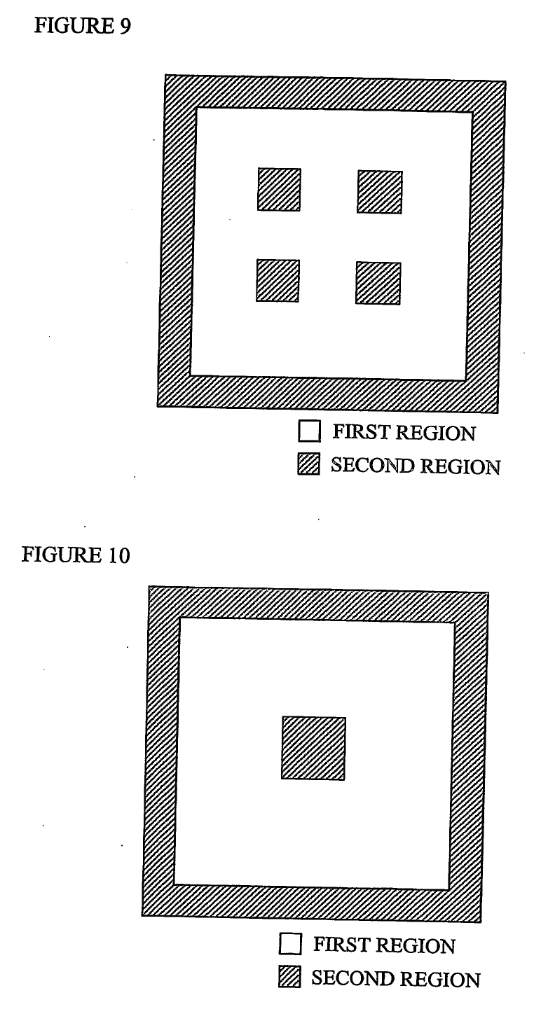

### REFERENCES

Driver J. and Dandekar A. M. (1997) Pest trap plants and crop protection. U.S. Pat. No. 5,640,804.
Romano C. P. (2002) Expression of Cry3B insecticidal protein in plants. U.S. Pat. No. 6,501,009.
Roush R. T. (1997) Bt-transgenic crops: just another pretty insecticide or a chance for a new start in resistance management. Pesticide science 51(3), 328-334.
Tang J. D., Collins H. L., Metz T. D., Earle E. D., Zhao J. Z., Roush R. T. and Shelton A. M. (2001) Greenhouse tests on resistance management of Bt transgenic plants using refuge strategies. Journal of Economic Entomology 94(1), 240-247.

1. A locus at which plant pests feed comprising at least two regions;
a) a first region comprises plants which produce at least a first pesticidal toxin; and
b) a second region comprises plants which produce at least a second pesticidal toxin; wherein a pest which can develop resistance to the first toxin does not develop resistance to the second toxin, and the first region comprises plants which produce the first toxin but not the second toxin when the plants of the second region produce the second toxin but not the first toxin.

2. A locus according to claim 1, wherein the plant pests are selected from the group consisting of insects, mites or nematodes.

3. A locus according to claim 2, wherein the plant pests are insects.

4. A locus according to claim 3 also comprising a third region, which region comprises non-insecticidal plants.

5. A locus according to claim 1, wherein the second region is within a mile from the first region.

6. A locus according to claim 5, wherein the second region is within a quarter of a mile from the first region.

7. A locus according to claim 6, wherein the second region is adjacent to the first region.

8. A locus according to claim 1, wherein the second region is a border around the perimeter of the first region.

9. A locus according to any of claim 1, wherein the second region comprises one or more strips within the first region.

10. A locus according to claim 1 to which comprises a random distribution of first and second regions.

11. A locus according to claim 1, wherein the first pesticidal toxin and the second pesticidal toxin are insecticidal toxins wherein said first insecticidal toxin has a different binding site to the second insecticidal toxin.

12. A locus according to any one of claim 1, wherein the first and second insecticidal toxin has a different mode of action to the second insecticidal toxin.

13. A locus according to any of claim 1, wherein the first insecticidal toxin is a crystal protein from Bacillus thuringiensis and the second insecticidal toxin is a VIP protein from Bacillus thuringiensis, or vice versa.

14. A locus according to claim 1, wherein the plants which comprise the first toxin and the plants which comprise the second toxin are from different genera.

15. A locus according to claim 1, wherein the plants which comprise the first toxin and the plants which comprise the second toxin are from the same genus.

16. A locus according to claim 1, wherein the plants which comprise the first toxin and the plants which comprise the second toxin are cotton plants.

17. A locus according to claim 1, wherein at least 5% of the locus comprises the first region and least 5% of the locus comprises the second region.

18. A locus according to claim 1, wherein at least 20% of the locus comprises the first region and at least 20% of the locus comprises the second region.

19. A locus according to claim 1, wherein 50% of the locus comprises the first region and 50% of the locus comprises the second region.

20. A method of controlling insects comprising providing a locus at which insects feed comprising at least two regions;
a) a first region comprising plants which produce at least a first insecticidal toxin; and
b) a second region comprising plants which produce at least a second insecticidal toxin; wherein an insect which can develop resistance to the first toxin does not develop resistance to the second toxin, and the first region comprises plants which produce the first toxin but not the second toxin when the plants of the second region produce the second toxin but not the first toxin.

21. (canceled)

22. A method of reducing the incidence of resistance of an insect to a first insecticidal toxin comprising the steps of providing a locus at which insects feed comprising at least two regions;
a) a first region comprising plants which produce at least a first insecticidal toxin; and
b) a second region comprising plants which produce at least a second insecticidal toxin; wherein an insect which can develop resistance to the first toxin does not develop resistance to the second toxin, and the first region comprises plants which produce the first toxin but not the second when the plants of the second region produce the second toxin but not the first, so that insects which have developed or are developing resistance to the first insecticidal toxin are controlled by the second toxin.

23. (canceled)

24. A method according to claim 20 wherein either the first or second region comprises Bollgard® cotton plants, expressing at least Cry I Ac.

25. A method according to claim 20 wherein the first region comprises Bollgard® cotton plants and the second region comprises VIP cotton plants, or vice versa.

26. A method according to claim 1 wherein the first region comprises plants which comprise Cry3A toxin from Bacillus thuringiensis or a modified version thereof, and the second region comprises plants which comprise Cry3B toxin from Bacillus thuringiensis.
